(19) United States
(12) Patent Application Publication
(10) Pub. No.: US 2025/0325203 A1
(43) Pub. Date: Oct. 23, 2025

(54) SYSTEM AND METHOD FOR EVALUATING CARDIAC PUMPING FUNCTION

(71) Applicant: Guy P. Curtis, San Diego, CA (US)

(72) Inventor: Guy P. Curtis, San Diego, CA (US)

(21) Appl. No.: 19/252,284

(22) Filed: Jun. 27, 2025

Related U.S. Application Data
(63) Continuation-in-part of application No. 17/580,300, filed on Jan. 20, 2022.

(51) Int. Cl. A61B 5/1455; A61B 5/00; A61B 5/02; A61B 5/026

(52) U.S. Cl. CPC A61B 5/14552; A61B 5/02028; A61B 5/0261; A61B 5/486; A61B 5/7275; A61B 5/7278

(57) ABSTRACT

An electronic device that evaluates a cardiac pumping function is described. This electronic device may perform the operations of: monitoring an instance of a pulse oximeter waveform of an individual; computing metric information associated with the instance of the pulse oximeter waveform; and calculating a rate of rise or fall of the instance of the pulse oximeter waveform as a function of time. In some embodiments, the electronic device may provide a recommended remedial action based at least in part on the calculated rate or rise of fall in the instance of the pulse oximeter waveform.

### CROSS REFERENCE TO RELATED APPLICATIONS

This application is a Continuation-in-Part of U.S. Non-Provisional application Ser. No. 17/580,300, “System and Method for Evaluating Cardiac Pumping Function,” filed on Jan. 20, 2022, by Guy P. Curtis, the contents of which are herein incorporated by reference.

### FIELD

The described embodiments relate to systems and methods for monitoring and evaluating a cardiac pumping function. More particularly, the described embodiments relate to systems and methods that evaluate cardiac pumping functions that are based on dynamic changes in blood pulse waveforms measured by an oximeter. The disclosed embodiments particularly, but not exclusively, useful for evaluating cardiac pumping functions by comparing the maximum second derivatives from a sequence of successive pulse oximeter waveforms, to assess the rise or fall of the waveforms as being indicative of the efficacy of the cardiac pumping function.

### BACKGROUND

A pulse oximeter waveform is often used as a graphical indication of the blood pressure response to a heart muscle function. Specifically, a pulse waveform shows the change in the amplitude A of blood pressure during a single contraction of the heart muscle. These waveforms are relatively short in duration and are, therefore, typically presented and considered as a continuous succession of pulse waveforms.

When considered individually each pulse waveform provides visual information of the velocity at which the amplitude A of the waveform is increasing or decreasing. Mathematically, this information is referred to as a first derivative, dA/dt. In addition to first derivative changes in velocity, pulses may also exhibit a rise or fall in amplitude of the entire waveform. This rise and/or fall of the waveform provides information about the acceleration of the waveform's amplitude A and is mathematically referred to as a second derivative, d²A/dt².

At the point of care, e.g., during surgery, information regarding changes in a heart muscle function can be quite helpful. Specifically, by monitoring a second derivative for the rise and/or fall of pulse waveforms, medical personnel can determine the beneficial or detrimental effect surgical activity may have had on heart muscle function. With this information, appropriate corrective action can be taken. In the event, it is usually obvious that corrective action, if needed, must be taken as soon as possible, or immediately.

### SUMMARY

An electronic device that evaluates a cardiac pumping function is described. This electronic device performs the operations of: monitoring an instance of a pulse oximeter waveform of an individual; computing metric information associated with the instance of the pulse oximeter waveform; and calculating a rate of rise or fall of the instance of the pulse oximeter waveform as a function of time.

Note that, during the monitoring, the electronic device may be coupled or attached to the individual, such as a patient.

Moreover, the rate of rise or fall may be expressed as a second derivative of an amplitude A of the instance of the pulse oximeter waveform, d²A/dt². Note that the second derivative of the amplitude of the instance of the pulse oximeter waveform may provide an early detection, from a single pulse waveform, of one or more trends for the overall heart muscle function.

Furthermore, in some embodiments, the electronic device may include a comparator, and the computing may be performed by the comparator. Notably, the comparator may compare a given instance of the pulse oximeter waveform (such as the instance of the pulse oximeter waveform) with an immediately preceding instance of the pulse oximeter waveform to calculate the rate of rise or fall.

Additionally, the electronic device may identify a maximum value of the second derivative and its location in the instance of the pulse oximeter waveform. This value and the location may be compared with another instance of the maximum value and the location obtained from a previous instance of the pulse oximeter waveforms. Based at least in part on this trend, the electronic device may evaluate a cardiac pumping function of the individual.

In some embodiments, the given instance of the pulse oximeter waveform may have a time interval that begins at a time tₒ and ends at a time tₑ. Multiple time segments Δt may be identified between tₒ and tₑ with each time segment Δt having a respective amplitude A. There may be two mathematical expressions of interest for describing a change in A (ΔA) with respect to each time segment. The first expression may be a velocity term, which may describe a change in the value of A as a function of time. Mathematically, this velocity term may be a first derivative, which may be expressed as dA/dt. Stated differently, in the context of the disclosed embodiments, the first derivative, dA/dt, may describe the slope or shape of the instance of the pulse oximeter waveform. The second expression of interest may be an acceleration term that describes a change of the velocity term as a function of time. Mathematically this acceleration term may be the second derivative, which may be expressed as d²A/dt². In the context of the disclosed embodiments, the second derivative, d²A/dt², may describe the rise and fall of the instance of the pulse oximeter waveform. As a practical consideration, it is the second derivative that may be indicative of blood flow volume and thus, the efficacy of the cardiac pumping function.

For the disclosed embodiments, the value and location of the maximum second derivative may be determined for each consecutive instance of the pulse oximeter waveform. The value and location for the maximum second derivative of each instance of the pulse oximeter waveform may then be compared with the value and location of the maximum second derivative in the immediately preceding instance of the pulse oximeter waveform. The purpose in the disclosed embodiments may be to determine a trend in the value of successive second derivatives for a comparative evaluation that may be used to determine the efficacy of a cardiac pumping function.

For the evaluation of the cardiac pumping function, the rise in the value of the second derivative may be indicative of improving function. On the other hand, a drop in the value of the second derivative may be indicative of a worsening function. Most likely the maximum value of the second derivative for each instance of the pulse oximeter waveform may occur during multiple time segments Δt immediately following tₒ. The disclosed embodiments envision the use of a visual display, in or associated with the electronic device, to show one or more trends in the maximum value of the second derivative, thereby determining or indicating the efficacy of the cardiac pumping function.

Moreover, in some embodiments, at least some of the aforementioned operations may be performed by a computer system (which may include at least a computer), which may be coupled to the electronic device. For example, the electronic device may provide, to the computer system, the instance of the pulse oximeter waveform and/or the metric information associated with the pulse oximeter waveform. Then, the computer system may perform at least some of the aforementioned operations.

Another embodiment provides an integrated circuit that performs at least some of the aforementioned operations.

Another embodiment provides a computer-readable storage medium for use with the electronic device or the computer system. When executed by the electronic device or the computer, this computer-readable storage medium causes the electronic device or the computer to perform at least some of the aforementioned operations.

Another embodiment provides a method, which may be performed by the electronic device or the computer system. This method includes at least some of the aforementioned operations.

This Summary is provided for purposes of illustrating some exemplary embodiments, so as to provide a basic understanding of some aspects of the subject matter described herein. Accordingly, it will be appreciated that the above-described features are examples and should not be construed to narrow the scope or spirit of the subject matter described herein in any way. Other features, aspects, and advantages of the subject matter described herein will become apparent from the following Detailed Description, Figures, and Claims.

### BRIEF DESCRIPTION OF THE FIGURES

FIG. 1 is a block diagram illustrating an example of communication between a monitoring device and an electronic device in accordance with an embodiment of the present disclosure.

FIG. 2 is a flow diagram illustrating an example of a method for evaluating cardiac pumping function in FIG. 1 in accordance with an embodiment of the present disclosure.

FIG. 3 is a drawing illustrating an example of components of a system that evaluates a cardiac pumping function in accordance with the present disclosure.

FIG. 4 is a drawing illustrating an example of a pulse oximeter waveform showing a mathematical first derivative expression for the velocity (or slope) of an instance of the pulse oximeter waveform of FIG. 3 in accordance with the present disclosure.

FIG. 5A is a drawing illustrating an example of a mathematical second derivative expression for the acceleration (or rise) of the instance of the pulse oximeter waveform of FIG. 1 in accordance with the present disclosure.

FIG. 5B is a drawing illustrating an example of a mathematical second derivative expression for the deceleration (or fall) of an instance of the pulse oximeter waveform of FIG. 3 in accordance with the present disclosure.

FIG. 6 is a drawing illustrating an example of the rise and fall of the instance of the pulse oximeter waveform of FIG. 3 resulting respectively from a positive second derivative (rise) and a negative second derivative (fall) in accordance with the present disclosure.

FIG. 7 is a drawing illustrating an example of maximum pressure slope in the aorta as a function of maximum pressure slope in the left ventricle in in a first run in accordance with the present disclosure.

FIG. 8 is a drawing illustrating an example of maximum pulse oximeter values as a function of maximum pressure slope in the aorta in the first run of FIG. 7 in accordance with the present disclosure.

FIG. 9 is a drawing illustrating an example of maximum pressure slope in the aorta as a function of maximum pressure slope in the left ventricle in in a second run accordance with the present disclosure.

FIG. 10 is a drawing illustrating an example of maximum pulse oximeter values as a function of maximum pressure slope in the aorta in the second run of FIG. 9 in accordance with the present disclosure.

FIG. 11 is a block diagram illustrating an example of an electronic device in accordance with an embodiment of the present disclosure.

Note that like reference numerals refer to corresponding parts throughout the drawings. Moreover, multiple instances of the same part are designated by a common prefix separated from an instance number by a dash.

### DETAILED DESCRIPTION

An electronic device that evaluates a cardiac pumping function is described. This electronic device may perform the operations of: monitoring an instance of a pulse oximeter waveform of an individual; computing metric information associated with the instance of the pulse oximeter waveform; and calculating a rate of rise or fall of the instance of the pulse oximeter waveform as a function of time. In some embodiments, the electronic device may provide a recommended remedial action based at least in part on the calculated rate or rise of fall in the instance of the pulse oximeter waveform.

By calculating the rate of rise or fall of the instance of the pulse oximeter waveform, these monitoring techniques may assess overall heart function (e.g., cardiac pumping function) of an individual. For example, changes in a maximum value of the rate of rise or fall of the instance of the pulse oximeter waveform as a function of time and a location of the maximum value may be indicative of changes in an efficacy of the cardiac pumping function. This dynamic and real-time assessment may be used by one or more individuals (e.g., at home, in an outpatient setting, etc.) and/or by one or more medical professionals (e.g., in a doctor's office, a hospital, an intensive-care unit, during surgery, etc.). Moreover, the monitoring techniques may provide a non-invasive assessment of the overall heart function of the individual. Furthermore, the monitoring techniques may enable or facilitate immediate (e.g., less than 1 min) corrective or remedial action, such as: biventricular pacing; adjustment of another intervention (such as a medication) based at least in part on the assessment provided by the monitoring techniques; and/or a recommendation to reposition one or more electrodes in an electrocardiogram (ECG) to an improved position or location on the individual. Additionally, the electronic device may be easier to use and may have a lower cost than existing approaches for cardiac monitoring (such as a Holter monitor, a cardiac monitor, e.g., a Zio patch from iRhythm Technologies, Inc., of San Francisco, California, an ECG, etc.). Consequently, the monitoring techniques may improve the quality and availability of assessments of overall heart function, may improve care of individuals and/or patients, and may reduce the cost of providing this care (e.g., by reducing in-patient evaluations and/or hospitalizations).

In the discussion that follows, electronic devices, computers and/or servers (which may be local or remotely located from each other) may communicate packets or frames in accordance with a wired communication protocol and/or a wireless communication protocol. The wireless communication protocol may include: a wireless communication protocol that is compatible with an Institute of Electrical and Electronics Engineers (IEEE) 802.11 standard (which is sometimes referred to as ‘Wi-Fi®,’ from the Wi-Fi Alliance of Austin, Texas), Bluetooth, Bluetooth low energy, a cellular-telephone network or data network communication protocol (such as a third generation or 3G communication protocol, a fourth generation or 4G communication protocol, e.g., Long Term Evolution or LTE (from the 3rd Generation Partnership Project of Sophia Antipolis, Valbonne, France), LTE Advanced or LTE-A, a fifth generation or 5G communication protocol, or other present or future developed advanced cellular communication protocol), and/or another type of wireless interface (such as another wireless-local-area-network interface). For example, an IEEE 802.11 standard may include one or more of: IEEE 802.11a, IEEE 802.11b, IEEE 802.11g, IEEE 802.11-2007, IEEE 802.11n, IEEE 802.11-2012, IEEE 802.11-2016, IEEE 802.11ac, IEEE 802.11ax, IEEE 802.11ba, IEEE 802.11be, or other present or future developed IEEE 802.11 technologies. Moreover, the wired communication protocol may include a wired communication protocol that is compatible with an IEEE 802.3 standard (which is sometimes referred to as ‘Ethernet’), e.g., an Ethernet II standard. However, a wide variety of communication protocols may be used. In the discussion that follows, Bluetooth and Ethernet are used as illustrative examples.

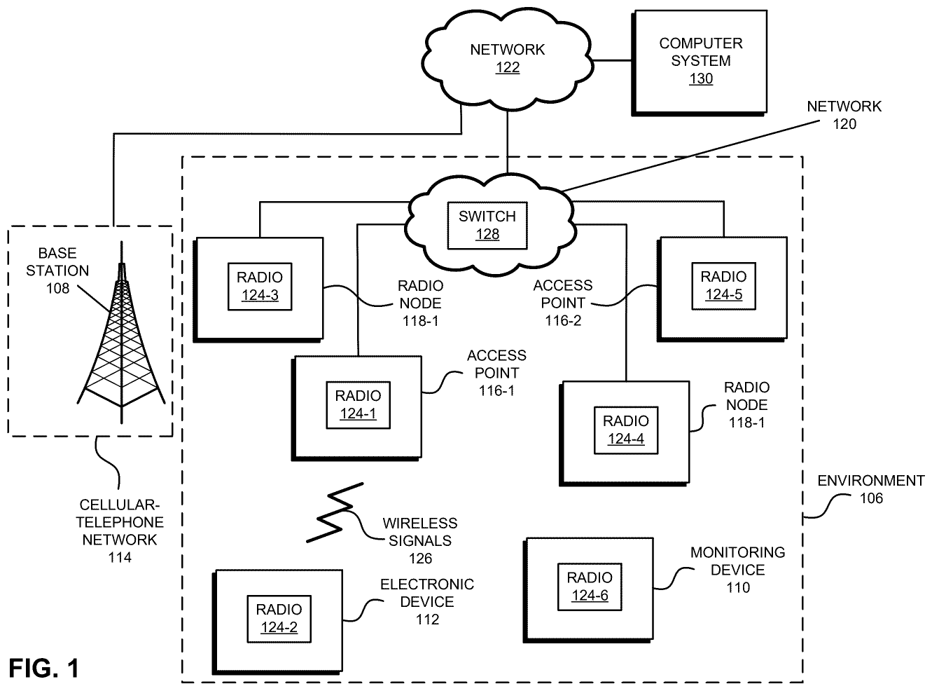

We now describe some embodiments of the monitoring techniques. FIG. 1 presents a block diagram illustrating an example of communication between a monitoring device 110 and an electronic device 112 (such as a cellular telephone, a portable electronic device, or another type of electronic device, etc.) in accordance with an embodiment of the present disclosure. Moreover, electronic device 112 may optionally communicate via a cellular-telephone network 114 (which may include a base station 108), one or more access points 116 (which may communicate using Wi-Fi) in a wireless local area network (WLAN) and/or radio node 118 (which may communicate using LTE) in a small-scale network (such as a small cell). For example, radio node 118 may include: an Evolved Node B (eNodeB), a Universal Mobile Telecommunications System (UMTS) NodeB and radio network controller (RNC), a New Radio (NR) gNB or gNodeB (which communicates with a network with a cellular-telephone communication protocol that is other than LTE), etc. In the discussion that follows, an access point, a radio node or a base station are sometimes referred to generically as a ‘communication device.’ Moreover, one or more base stations (such as base station 108), access points 116, and/or radio node 118 may be included in one or more networks, such as: a WLAN, a small cell, a local area network (LAN) and/or a cellular-telephone network. In some embodiments, access points 116 may include a physical access point and/or a virtual access point that is implemented in software in an environment of an electronic device or a computer.

Furthermore, electronic device 112 may optionally communicate with computer system 130 (which may include one or more computers or servers, and which may be implemented locally or remotely to provide storage and/or analysis services) using a wired communication protocol (such as Ethernet) via network 120 and/or 122. Note that networks 120 and 122 may be the same or different networks. For example, networks 120 and/or 122 may be a LAN, an intra-net or the Internet. In some embodiments, the wired communication protocol may include a secured connection over transmission control protocol/Internet protocol (TCP/IP) using hypertext transfer protocol secure (HTTPS) with a JavaScript object notation (JSON) Web services connection. Additionally, in some embodiments, network 120 may include one or more routers and/or switches (such as switch 128).

In some embodiments, electronic device 112 and/or computer system 130 may optionally implement at least some of the operations in the monitoring techniques. Notably, as described further below, electronic device 112 and/or computer system 130 may optionally perform at least some of the analysis of measurement data acquired by monitoring device 110, and may optionally provide feedback information to monitoring device 110.

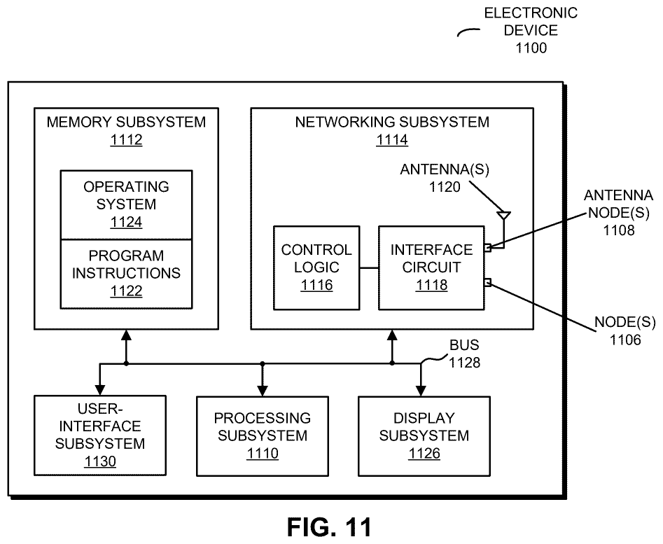

As described further below with reference to FIG. 11, base station 108, monitoring device 110, electronic device 112, access points 116, radio node 118, switch 128 and/or computer system 130 may include subsystems, such as a networking subsystem, a memory subsystem and a processor subsystem. In addition, monitoring device 110, electronic device 112, access points 116 and radio node 118 may include radios 124 in the networking subsystems. More generally, monitoring device 110, electronic device 112, access points 116 and radio node 118 can include (or can be included within) any electronic devices with the networking subsystems that enable monitoring device 110, electronic device 112, access points 116 and radio node 118 to wirelessly communicate with one or more other electronic devices. This wireless communication can comprise transmitting access on wireless channels to enable electronic devices to make initial contact with or detect each other, followed by exchanging subsequent data/management frames (such as connection requests and responses) to establish a connection, configure security options, transmit and receive frames or packets via the connection, etc.

During the communication in FIG. 1, base station 108, monitoring device 110, electronic device 112, access points 116, radio node 118 and/or computer system 130 may wired or wirelessly communicate while: transmitting access requests and receiving access responses on wired or wireless channels, detecting one another by scanning wireless channels, establishing connections (for example, by transmitting connection requests and receiving connection responses), and/or transmitting and receiving frames or packets (which may include information as payloads).

As can be seen in FIG. 1, wireless signals 126 (represented by a jagged line) may be transmitted by radios 124 in, e.g., access points 116 and/or radio node 118 and monitoring device 110 or electronic device 112. For example, radio 124-1 in access point 116-1 may transmit information (such as one or more packets or frames) using wireless signals 126. These wireless signals are received by radio 124-2 in electronic device 112. This may allow access point 116-1 to communicate information to other access points 116 and/or electronic device 112. Note that wireless signals 126 may convey one or more packets or frames.

In the described embodiments, processing a packet or a frame in one or more electronic devices in monitoring device 110, electronic device 112, access points 116, radio node 118 and/or computer system 130 may include: receiving the wireless or electrical signals with the packet or the frame; decoding/extracting the packet or the frame from the received wireless or electrical signals to acquire the packet or the frame; and processing the packet or the frame to determine information contained in the payload of the packet or the frame.

Note that the wired and/or wireless communication in FIG. 1 may be characterized by a variety of performance metrics, such as: a data rate for successful communication (which is sometimes referred to as ‘throughput’), an error rate (such as a retry or resend rate), a mean-squared error of equalized signals relative to an equalization target, intersymbol interference, multipath interference, a signal-to-noise ratio, a width of an eye pattern, a ratio of number of bytes successfully communicated during a time interval (such as 1-10 s) to an estimated maximum number of bytes that can be communicated in the time interval (the latter of which is sometimes referred to as the ‘capacity’ of a communication channel or link), and/or a ratio of an actual data rate to an estimated data rate (which is sometimes referred to as ‘utilization’). While instances of radios 124 are shown in components in FIG. 1, one or more of these instances may be different from the other instances of radios 124.

In some embodiments, wireless communication between components in FIG. 1 uses one or more bands of frequencies, such as: 900 MHz, 2.4 GHz, 5 GHz, 6 GHz, 7 GHz, 60 GHz, the Citizens Broadband Radio Spectrum or CBRS (e.g., a frequency band near 3.5 GHz), and/or a band of frequencies used by LTE or another cellular-telephone communication protocol or a data communication protocol. Note that the communication between electronic devices may use multi-user transmission (such as orthogonal frequency division multiple access or OFDMA) and/or multiple-input multiple-output (MIMO).

Although we describe the network environment shown in FIG. 1 as an example, in alternative embodiments, different numbers or types of electronic devices may be present. For example, some embodiments comprise more or fewer electronic devices. As another example, in another embodiment, different electronic devices are transmitting and/or receiving packets or frames.

While FIG. 1 illustrates computer system 130 at a particular location, in other embodiments at least a portion of computer system 130 is implemented at more than one location. Thus, in some embodiments, computer system 130 is implemented in a centralized manner, while in other embodiments at least a portion of computer system 130 is implemented in a distributed manner.

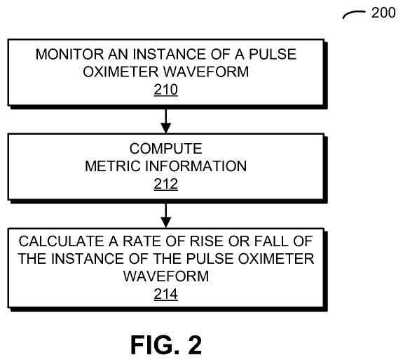

As described further below with reference to FIGS. 2-10, in order to address these challenges, a monitoring device 110 (such as a pulse oximeter) may be remateably attached to or coupled to an individual. Monitoring device 110 may collect or measure one or more instances of pulse oximetry waveforms. Then, monitoring device 110 may analyze the one or more instances of pulse oximetry waveforms to: compute metric information associated with the one or more instances of the pulse oximeter waveform; and/or calculate a rate of rise or fall of the one or more instances of the pulse oximeter waveform as a function of time

Alternatively or additionally, monitoring device 110 may provide, to electronic device 112 and/or computer system 130, the one or more instances of the pulse oximeter waveform and/or metric information associated with the one or more instances of the pulse oximeter waveform. Then, electronic device 112 and/or computer system 130 may perform at least some of the aforementioned operations, such as the analysis.

In these ways, the monitoring techniques may facilitate dynamic and real-time monitoring of cardiac pumping function of the individual. Because the monitoring is non-invasive, the monitoring techniques may be easier to use and may have a lower cost than existing approaches for cardiac monitoring. Moreover, the monitoring techniques may enable or facilitate immediate (e.g., less than 1 min) corrective or remedial action. Consequently, the monitoring techniques may improve the quality and availability of assessments of overall heart function, may improve care of individuals and/or patients, and may reduce the cost of providing this care (e.g., by reducing in-patient evaluations and/or hospitalizations).

We now describe embodiments of the method. FIG. 2 presents a flow diagram illustrating an example of a method 200 for evaluating cardiac pumping function. This method may be performed by an electronic device, such as monitoring device 110.

During operation, the electronic device may monitor an instance of a pulse oximeter waveform (operation 210) of an individual. Then, the electronic device may optionally compute metric information (operation 212) associated with the instance of the pulse oximeter waveform. Next, the electronic device may calculate a rate of rise or fall of the instance of the pulse oximeter waveform (operation 214) as a function of time. For example, the calculation (operation 214) may be based at least in part on the instance of the pulse oximeter waveform and/or the metric information.

Note that, during the monitoring (operation 210), the electronic device may be coupled or attached to the individual, such as a patient.

Moreover, the rate of rise or fall may be expressed as a second derivative of an amplitude A of the instance of the pulse oximeter waveform, d²A/dt². Note that the second derivative of the amplitude of the instance of the pulse oximeter waveform may provide an early detection, from a single pulse waveform, of one or more trends for the overall heart muscle function.

Furthermore, in some embodiments, the electronic device may include a comparator, and the computing (operation 212) may be performed by the comparator. Notably, the comparator may compare a given instance of the pulse oximeter waveform (such as the instance of the pulse oximeter waveform) with an immediately preceding instance of the pulse oximeter waveform to calculate the rate of rise or fall (operation 214).

Additionally, the electronic device may identify a maximum value of the second derivative and its location in the instance of the pulse oximeter waveform. This value and the location may be compared with another instance of the maximum value and the location obtained from a previous instance of the pulse oximeter waveforms. Based at least in part on this trend, the electronic device may evaluate a cardiac pumping function of the individual.

In some embodiments, the given instance of the pulse oximeter waveform may have a time interval that begins at a time tₒ and ends at a time tₑ. Multiple time segments Δt may be identified between tₒ and tₑ with each time segment Δt having a respective amplitude A. There may be two mathematical expressions of interest for describing a change in A (ΔA) with respect to each time segment. The first expression may be a velocity term, which may describe a change in the value of A as a function of time. Mathematically, this velocity term may be a first derivative, which may be expressed as dA/dt. Stated differently, in the context of the disclosed embodiments, the first derivative, dA/dt, may describe the slope or shape of the instance of the pulse oximeter waveform. The second expression of interest may be an acceleration term that describes a change of the velocity term as a function of time. Mathematically this acceleration term may be the second derivative, which may be expressed as d²A/dt². In the context of the disclosed embodiments, the second derivative, d²A/dt², may describe the rise and fall of the instance of the pulse oximeter waveform. As a practical consideration, it is the second derivative that may be indicative of blood flow volume and thus, the efficacy of the cardiac pumping function.

For the disclosed embodiments, the value and location of the maximum second derivative may be determined for each consecutive instance of the pulse oximeter waveform. The value and location for the maximum second derivative of each instance of the pulse oximeter waveform may then be compared with the value and location of the maximum second derivative in the immediately preceding instance of the pulse oximeter waveform. The purpose in the disclosed embodiments may be to determine a trend in the value of successive second derivatives for a comparative evaluation that may be used to determine the efficacy of a cardiac pumping function.

For the evaluation of the cardiac pumping function, the rise in the value of the second derivative may be indicative of improving function. On the other hand, a drop in the value of the second derivative may be indicative of a worsening function. Most likely the maximum value of the second derivative for each instance of the pulse oximeter waveform may occur during multiple time segments Δt immediately following tₒ. The disclosed embodiments envision the use of a visual display, in or associated with the electronic device, to show one or more trends in the maximum value of the second derivative, thereby determining or indicating the efficacy of the cardiac pumping function.

In some embodiments of method 200, there may be additional or fewer operations. Furthermore, the order of the operations may be changed, and/or two or more operations may be combined into a single operation.

We now further describe detection of the monitoring techniques. In the discussion that follows, the monitoring techniques are illustrated using a pulse oximeter transmits or provides one or more instances of pulse oximeter waveforms to a computer for analysis.

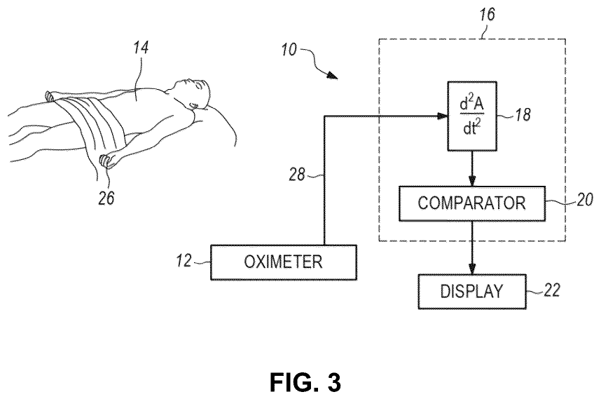

FIG. 3 presents a drawing illustrating an example of components of a system 10 that evaluates a cardiac pumping function. As shown, system 10 includes an oximeter 12 that can be connected with a patient 14 (and, more generally, an individual) for the purpose of monitoring blood flow characteristics of patient 14. FIG. 3 also shows that system 10 may include a computer 16 which is attached to oximeter 12, and that computer 16 may include a differentiator 18 and a comparator 20. A display 22 may present clinical results of measurements from oximeter 12 that are pertinent to the blood flow characteristics of patient 14. Notably, these blood flow characteristics may be based at least in part on measurements of an instance of a pulse oximeter waveform 24 (see FIG. 2) that is obtained by oximeter 12.

Operationally, oximeter 12 may be typically connected with a finger 26 of patient 14 to measure and record the physical characteristics of the instance of the patient's pulse oximeter waveform 24. The obtained measurements may then be transmitted as metric information to computer 16 via an electronic connection 28. In the disclosed monitoring techniques mathematical expressions may be based at least in part on this metric information. Specifically, these mathematical expressions may include first and second derivatives which may be generated by differentiator 18 in computer 16. Note that the mathematical expressions may be pertinent to changes in the instance of pulse oximeter waveform 24.

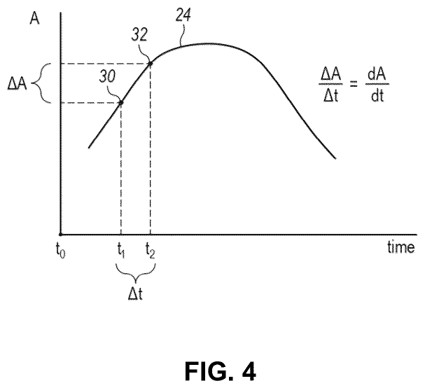

FIG. 4 presents a drawing illustrating an example of a pulse oximeter waveform showing a mathematical first derivative expression for the velocity (or slope) of an instance of the pulse oximeter waveform of FIG. 3. Notably, FIG. 4 presents a portion of the instance of pulse oximeter waveform 24 whereon a change in the amplitude A of the instance of pulse oximeter waveform 24 is shown as a function of time. Mathematically, such a change may be expressed as ΔA/Δt (or dA/dt). This expression is sometimes referred to as a ‘first derivative,’ which may establish the ‘slope’ of the instance of pulse oximeter waveform 24. The expression ΔA/Δt, or dA/dt, is also sometimes referred to as the ‘velocity’ of waveform 24.

Graphically, a change in the amplitude, ΔA, of the instance of pulse oximeter waveform 24 is shown in FIG. 4 to occur between points 30 and 32 during the time interval Δt between t₁ and t₂. In some embodiments of the disclosed monitoring techniques, this first derivative dA/dt, that occurs during the time interval t₁ to t₂, may be used by the comparator 20 of computer 16 for comparison with the first derivative of the instance of pulse oximeter waveform 24 during the immediately subsequent same time interval Δt between t₂ and t₃. As disclosed below, this comparison may be performed to determine an acceleration of amplitude A of the instance of pulse oximeter waveform 24.

Another mathematical expression of interest for the disclosed monitoring techniques is the second derivative of the instance of pulse oximeter waveform 24, d²A/dt². This derivative may express the time rate of change of the first derivative. It is sometimes referred to as the ‘acceleration’ of the instance of pulse oximeter waveform 24. This second derivative, or acceleration, may be used by system 10 as it mathematically expresses the rise and/or fall of the instance of pulse oximeter waveform 24 as a function of time. Stated differently, as a practical consequence, the rise and fall of the instance of pulse oximeter waveform 24 may be indicative of the volume of blood flow; with a rise being indicative of improved blood flow for patient 14, and a fall (or drop) being indicative of a worsening of his/her blood flow condition.

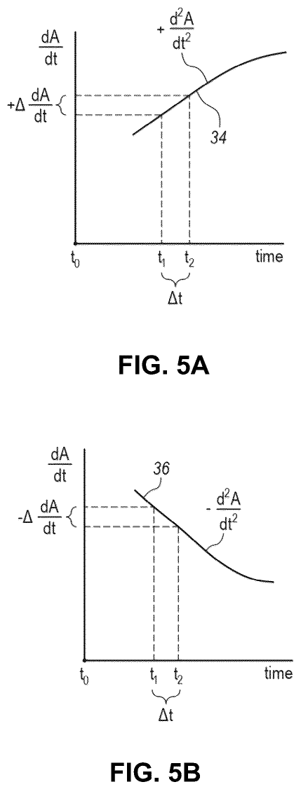

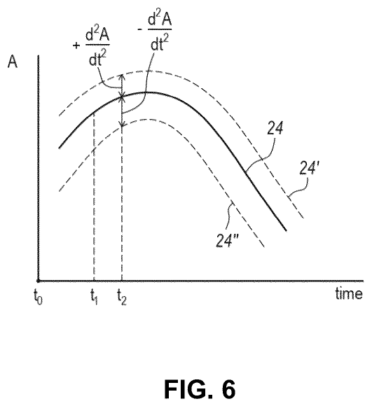

FIG. 5A presents a drawing illustrating an example of a mathematical second derivative expression for the acceleration (or rise) of the instance of the pulse oximeter waveform of FIG. 1. Moreover, FIG. 5B presents a drawing illustrating an example of a mathematical second derivative expression for the deceleration (or fall) of an instance of the pulse oximeter waveform of FIG. 3. In FIG. 5A, line curve 34 may represent an increasing second derivative (positive d²A/dt²), which indicates an acceleration in the magnitude of A. On the other hand, line curve 36 in FIG. 5B may represent a decreasing second derivative (negative d²A/dt²), which may indicate a deceleration in the magnitude of A. The consequences of these accelerations and decelerations are shown below in FIG. 6.

FIG. 6 presents a drawing illustrating an example of the rise and fall of the instance of the pulse oximeter waveform of FIG. 3 resulting respectively from a positive second derivative (rise) and a negative second derivative (fall). The instance of pulse oximeter waveform 24 shown in FIG. 6 may be representative of a constant instance of pulse oximeter waveform 24 in which the amplitude A of the instance of pulse oximeter waveform 24 has neither accelerated nor decelerated. However, when comparator 20 in computer 16 detects a second derivative change in the amplitude A, display 22 in the system may present a visual indication of the change. Specifically, as shown in FIG. 6 an acceleration may be shown on display 22 as a movement of instance of pulse oximeter waveform 24 toward a raised position shown for an instance of pulse oximeter waveform 24′. A deceleration, however, may show on display 22 as a movement of instance of pulse oximeter waveform 24 toward a lower position shown for an instance of pulse oximeter waveform 24″. As noted previously, these movements may provide valuable information to a healthcare provider (such as a physician) at the time of care for an immediate response, when needed.

In some embodiments, at least some of the operations in the monitoring techniques may be performed using a pretrained analysis model. For example, the pretrained analysis model may include a neural network. More generally, a variety of supervised-learning techniques may be used to generate the pretrained model. For example, the supervised-learning techniques may include: a linear model, a nonlinear model, Support Vector Machines, LASSO, etc.

We now describe recorded observations of a sequence of events following coronary artery occlusion to cardiac muscle downstream. These observations provide an assessment of the earliest and most sensitive measures of function and malfunction. Notably, pig hearts were instrumented with ultrasonic crystals so that small regions of contracting muscle or shortening could be monitored, blood pressure and ECGs were simultaneously recorded. The artery subtending the area where shortening was being monitored was suddenly occluded and within about 5 s shortening began to be progressively diminished. ECG changes indicating this event did not occur until about one minute after the occlusion. Changes in blood pressure did not occur early on. The conclusion was that regional contractility was the most sensitive measure of regional perfusion and performance. Global ejection fraction was not measured in these experiments, but other work suggests it would lag behind these regional changes in shortening. However, dP/dT max for left ventricular pressure would predictably be changing early reflecting these regional changes.

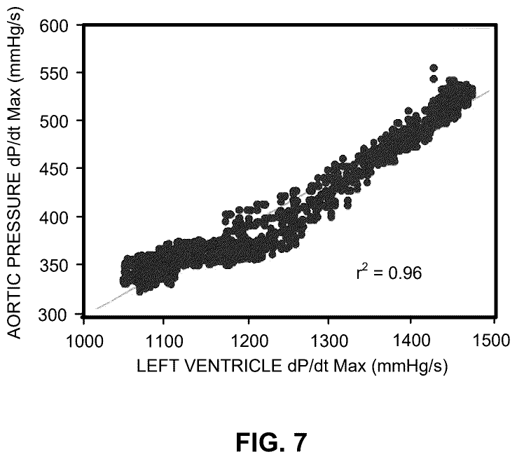

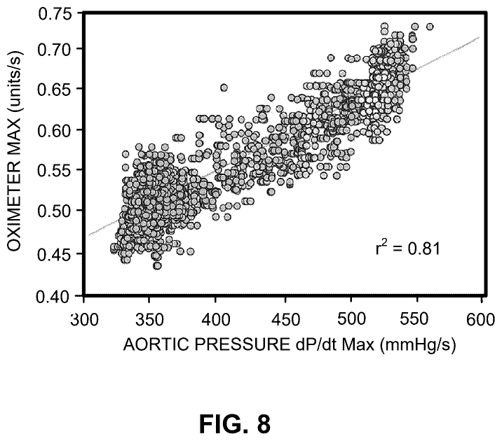

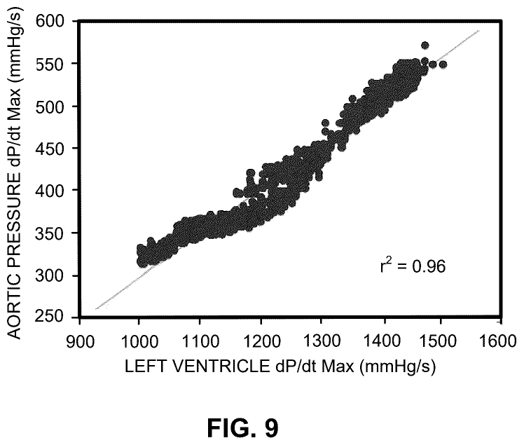

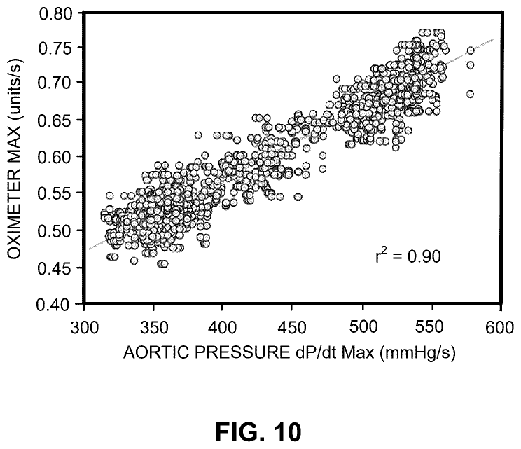

Moreover, in some embodiments of the measurements on pigs, two runs were captured in which pressures were measured simultaneously in the left ventricle, the aorta and the pulse oximeter reading from the tongue. FIGS. 7 and 9 are drawings illustrating examples of maximum pressure slope in the aorta as a function of maximum pressure slope in the left ventricle in in a first run or a second run. Moreover, FIGS. 8 and 10 are drawings illustrating an example of maximum pulse oximeter values as a function of maximum pressure slope in the aorta in the first run of FIG. 7 or the second run of FIG. 9. Notably, FIGS. 7-10 shown simultaneous matched beats plotted for the maximum rate of rise of the waveform from left ventricle (LV), aorta (Ao) and the raw pulse oximeter waveform output from the oximeter (Pox). Note that there is good agreement for the arterial waveform and the pulse oximeter waveform with the left ventricular pressure.

These monitoring techniques may be used in a wide variety of settings to noninvasively measure pump function in a human or an animal, such as a race horse, a cat, a dog, an athlete, a patient at varying stage of wellness, etc.

The monitoring techniques may provide an extremely sensitive way to monitor heart function. For example, by monitoring a rate of rise or fall of an instance of a pulse oximetry waveform, the monitoring techniques may provide a noninvasive way to monitor a rate of rise or fall of blood pressure in the left ventricle and/or blood pressure in a peripheral artery, such as: the aorta, a brachial artery, a radial artery, etc. Thus, the monitoring techniques may provide a sensitive way to measure cardiac function (e.g., the monitoring techniques may be more sensitive to a change in cardiac function than blood pressure). In some embodiments, by detecting changes when an individual exercises (such as small changes in regional function), the monitoring techniques may allow disease or changes associated with disease to be more easily detected. Notably, when an artery is occluded, a change may be detected within 5 s (as opposed to approximately 1 min. with an ECG). This may result in a significant improvement in the efficiency of an electronic device or a computer system that implements the monitoring techniques, such as: reduced processor cycles, reduced memory consumption, reduced network bandwidth to communicate data, etc.

Note that an ejection fraction may be a static value corresponding to a difference of the systolic pressure and the diastolic pressure. Moreover, the ejection fraction may be a global measurement. In contrast, the monitoring techniques may provide a more sensitive, local measurement of the total efficacy of muscle contraction of the heart. For example, the monitoring techniques may facilitate continuous, periodic or as-needed monitoring in a doctor's office, a hospital or at home relative to a baseline (which may have an unknown duration). In some embodiments, the monitoring techniques may use optical (e.g., red) light and/or infrared light, such as: light having wavelengths between 620-750 nm and/or between 700 nm-1 mm.

The noninvasive monitoring techniques may be used in: an operating room, in an intensive care unit, and/or to monitor a patient with heart disease (e.g., at home). Moreover the monitoring techniques may be used by a medical professional (such as a physician) or by a lay-person.

While the preceding discussion illustrated the monitoring techniques using a second derivative of the instance of the pulse oximeter waveform or measurements, in other embodiments a first derivative of the instance of the pulse oximeter waveform is used. Moreover, in some embodiments, the second derivative of the instance of the pulse oximeter waveform is used when a first derivative of the instance of the pulse oximeter waveform is approximately constant (such as within 5-10% of a baseline value, e.g., a value in a preceding time interval, such as 30 s, 1 min., 5 min, 10 min. or 30 min.).

Note that the first derivative of the pressure waveform may be used because it may be most affected by the pumping action of the heart muscle and less affected by other parts of the circulatory system. It may measure the rate of pressure generation or muscle shortening from the time the muscle is stimulated to contract and the mitral valve closes, until the aortic valve opens and blood begins to escape the heart (which is sometimes referred to as ‘the period of isovolumic contraction’). In general, the things that can affect this event may be: a change in the muscle's ability to contract, an increase in blood volume returning to the heart, or a fall in the diastolic pressure. When diastolic pressure is stable and venous return to the heart is stable then changes in contractility can be very sensitively measured.

Furthermore, note that the second derivative of the pulse oximeter waveform may be very dependent on the entire circulatory system, such as: changes in volume in the circulatory system, or changes in vascular resistance or pump function. Such changes can influence the instance of the pulse oximeter waveform quite dramatically and correct analysis of the variable that is changing can be challenging. Consequently, analysis of one or more instances of the pulse oximeter waveform may involve use of a predefined analysis model (such as a neural network or a machine-learning model) that determines blood pressure from the blood flow signal measured by the pulse oximeter.

In the present discussion, ventricular dP/dT may be a rate of pressure change with time during isovolemic contraction of the cardiac ventricles, e.g., in the period before the aortic valve and/or pulmonic valve opens, when there is no considerable change in left atrial or right atrial pressure. It may be a noninvasive technique for measuring the contractility of the left ventricle using pulse oximetry. However, in other embodiments, echocardiography may be used instead of or in addition to pulse oximetry.

In some embodiments, dP/dt may be used to predict postoperative systolic function in an individual with mitral valve regurgitation. However, the disclosed monitoring techniques may be used to monitor a variety of cardiac conditions.

For example, a continuous-wave Doppler velocity waveform of a mitral regurgitation jet may be determined from an apical four-chamber view. This may enable the measurement of instantaneous pressure gradients between the left ventricle and the left atrium. The left ventricle dP/dt may be calculated using an interval of 1-3 m/sec on the mitral regurgitation velocity spectrum.

The rate of pressure change in the right ventricle may be calculated from the tricuspid regurgitation in the same way as in the left ventricle using the mitral regurgitation jet. The exception may be that, on the right side, an interval of 1-2 m/s may be used.

Note that dP/dt may denote a rate at which left ventricular pressure rises and may indicate systolic contractile function. For example, for the left ventricle, normal may be greater than 1200 mm Hg/s, borderline may be 800-1200 mm Hg/s, reduced may be less than 800 mm Hg/s, and severely reduced may be less than 500 mm Hg/s. Alternatively, for the right ventricle, normal may be greater than 400 mm Hg/s.

We now describe embodiments of a monitoring device, an electronic device, or a computer in a computer system, which may perform at least some of the operations in the monitoring techniques. FIG. 11 presents a block diagram illustrating an example of an electronic device 1100, e.g., monitoring device 110, electronic device 112, access points 116, radio node 118, switch 128 and/or a computer or server in computer system 130, in accordance with some embodiments. For example, electronic device 1100 may include: processing subsystem 1110, memory subsystem 1112, and networking subsystem 1114. Processing subsystem 1110 includes one or more devices configured to perform computational operations. For example, processing subsystem 1110 can include one or more microprocessors, ASICs, microcontrollers, programmable-logic devices, GPUs and/or one or more DSPs. Note that a given component in processing subsystem 1110 are sometimes referred to as a ‘computation device’.

Memory subsystem 1112 includes one or more devices for storing data and/or instructions for processing subsystem 1110 and networking subsystem 1114. For example, memory subsystem 1112 can include dynamic random access memory (DRAM), static random access memory (SRAM), and/or other types of memory. In some embodiments, instructions for processing subsystem 1110 in memory subsystem 1112 include: program instructions or sets of instructions (such as program instructions 1122 or operating system 1124), which may be executed by processing subsystem 1110. Note that the one or more computer programs or program instructions may constitute a computer-program mechanism. Moreover, instructions in the various program instructions in memory subsystem 1112 may be implemented in: a high-level procedural language, an object-oriented programming language, and/or in an assembly or machine language. Furthermore, the programming language may be compiled or interpreted, e.g., configurable or configured (which may be used interchangeably in this discussion), to be executed by processing subsystem 1110.

In addition, memory subsystem 1112 can include mechanisms for controlling access to the memory. In some embodiments, memory subsystem 1112 includes a memory hierarchy that comprises one or more caches coupled to a memory in electronic device 1100. In some of these embodiments, one or more of the caches is located in processing subsystem 1110.

In some embodiments, memory subsystem 1112 is coupled to one or more high-capacity mass-storage devices (not shown). For example, memory subsystem 1112 can be coupled to a magnetic or optical drive, a solid-state drive, or another type of mass-storage device. In these embodiments, memory subsystem 1112 can be used by electronic device 1100 as fast-access storage for often-used data, while the mass-storage device is used to store less frequently used data.

Networking subsystem 1114 includes one or more devices configured to couple to and communicate on a wired and/or wireless network (i.e., to perform network operations), including: control logic 1116, an interface circuit 1118 and one or more antennas 1120 (or antenna elements). (While FIG. 11 includes one or more antennas 1120, in some embodiments electronic device 1100 includes one or more nodes, such as antenna nodes 1108, e.g., a metal pad or a connector, which can be coupled to the one or more antennas 1120, or nodes 1106, which can be coupled to a wired or optical connection or link. Thus, electronic device 1100 may or may not include the one or more antennas 1120. Note that the one or more nodes 1106 and/or antenna nodes 1108 may constitute input(s) to and/or output(s) from electronic device 1100.) For example, networking subsystem 1114 can include a Bluetooth™ networking system, a cellular networking system (e.g., a 3G/4G/5G network such as UMTS, LTE, etc.), a USB networking system, a networking system based on the standards described in IEEE 802.11 (e.g., a Wi-Fi® networking system), an Ethernet networking system, and/or another networking system.

Networking subsystem 1114 includes processors, controllers, radios/antennas, sockets/plugs, and/or other devices used for coupling to, communicating on, and handling data and events for each supported networking system. Note that mechanisms used for coupling to, communicating on, and handling data and events on the network for each network system are sometimes collectively referred to as a ‘network interface’ for the network system. Moreover, in some embodiments a ‘network’ or a ‘connection’ between electronic devices does not yet exist. Therefore, electronic device 1100 may use the mechanisms in networking subsystem 1114 for performing simple wireless communication between electronic devices, e.g., transmitting advertising or beacon frames and/or scanning for advertising frames transmitted by other electronic devices.

Within electronic device 1100, processing subsystem 1110, memory subsystem 1112, and networking subsystem 1114 are coupled together using bus 1128. Bus 1128 may include an electrical, optical, and/or electro-optical connection that the subsystems can use to communicate commands and data among one another. Although only one bus 1128 is shown for clarity, different embodiments can include a different number or configuration of electrical, optical, and/or electro-optical connections among the subsystems.

In some embodiments, electronic device 1100 includes a display subsystem 1126 for displaying information on a display, which may include a display driver and the display, such as a liquid-crystal display, a multi-touch touchscreen, etc. Moreover, electronic device 1100 may include a user-interface subsystem 1130, such as: a mouse, a keyboard, a trackpad, a stylus, a voice-recognition interface, and/or another human-machine interface.

Electronic device 1100 can be (or can be included in) any electronic device with at least one network interface. For example, electronic device 1100 can be (or can be included in): a desktop computer, a laptop computer, a subnotebook/netbook, a server, a supercomputer, a tablet computer, a smartphone, a smartwatch, a pulse oximeter, a cellular telephone, a consumer-electronic device, a portable computing device, communication equipment, a monitoring device and/or another electronic device.

Although specific components are used to describe electronic device 1100, in alternative embodiments, different components and/or subsystems may be present in electronic device 1100. For example, electronic device 1100 may include one or more additional processing subsystems, memory subsystems, networking subsystems, and/or display subsystems. Additionally, one or more of the subsystems may not be present in electronic device 1100. Moreover, in some embodiments, electronic device 1100 may include one or more additional subsystems that are not shown in FIG. 11. Also, although separate subsystems are shown in FIG. 11, in some embodiments some or all of a given subsystem or component can be integrated into one or more of the other subsystems or component(s) in electronic device 1100. For example, in some embodiments program instructions 1122 are included in operating system 1124 and/or control logic 1116 is included in interface circuit 1118.

Moreover, the circuits and components in electronic device 1100 may be implemented using any combination of analog and/or digital circuitry, including: bipolar, PMOS and/or NMOS gates or transistors. Furthermore, signals in these embodiments may include digital signals that have approximately discrete values and/or analog signals that have continuous values. Additionally, components and circuits may be single-ended or differential, and power supplies may be unipolar or bipolar.

An integrated circuit may implement some or all of the functionality of networking subsystem 1114 and/or electronic device 1100. The integrated circuit may include hardware and/or software mechanisms that are used for transmitting signals from electronic device 1100 and receiving signals at electronic device 1100 from other electronic devices. Aside from the mechanisms herein described, radios are generally known in the art and hence are not described in detail. In general, networking subsystem 1114 and/or the integrated circuit may include one or more radios.

In some embodiments, an output of a process for designing the integrated circuit, or a portion of the integrated circuit, which includes one or more of the circuits described herein may be a computer-readable medium such as, for example, a magnetic tape or an optical or magnetic disk or solid state disk. The computer-readable medium may be encoded with data structures or other information describing circuitry that may be physically instantiated as the integrated circuit or the portion of the integrated circuit. Although various formats may be used for such encoding, these data structures are commonly written in: Caltech Intermediate Format (CIF), Calma GDS II Stream Format (GDSII), Electronic Design Interchange Format (EDIF), OpenAccess (OA), or Open Artwork System Interchange Standard (OASIS). Those of skill in the art of integrated circuit design can develop such data structures from schematics of the type detailed above and the corresponding descriptions and encode the data structures on the computer-readable medium. Those of skill in the art of integrated circuit fabrication can use such encoded data to fabricate integrated circuits that include one or more of the circuits described herein.

While some of the operations in the preceding embodiments were implemented in hardware or software, in general the operations in the preceding embodiments can be implemented in a wide variety of configurations and architectures. Therefore, some or all of the operations in the preceding embodiments may be performed in hardware, in software or both. For example, at least some of the operations in the monitoring techniques may be implemented using program instructions 1122, operating system 1124 (such as a driver for interface circuit 1118) or in firmware in interface circuit 1118. Thus, the monitoring techniques may be implemented at runtime of program instructions 1122. Alternatively or additionally, at least some of the operations in the monitoring techniques may be implemented in a physical layer, such as hardware in interface circuit 1118.

In the preceding description, we refer to ‘some embodiments.’ Note that ‘some embodiments’ describes a subset of all of the possible embodiments, but does not always specify the same subset of embodiments. Moreover, note that the numerical values provided are intended as illustrations of the monitoring techniques. In other embodiments, the numerical values can be modified or changed.

The foregoing description is intended to enable any person skilled in the art to make and use the disclosure, and is provided in the context of a particular application and its requirements. Moreover, the foregoing descriptions of embodiments of the present disclosure have been presented for purposes of illustration and description only. They are not intended to be exhaustive or to limit the present disclosure to the forms disclosed. Accordingly, many modifications and variations will be apparent to practitioners skilled in the art, and the general principles defined herein may be applied to other embodiments and applications without departing from the spirit and scope of the present disclosure. Additionally, the discussion of the preceding embodiments is not intended to limit the present disclosure. Thus, the present disclosure is not intended to be limited to the embodiments shown, but is to be accorded the widest scope consistent with the principles and features disclosed herein.

What is claimed is:

1. An integrated circuit, wherein the integrated circuit is configured to monitor an instance of a pulse oximeter waveform of an individual, wherein the integrated circuit is configured to:
monitor the instance of a pulse oximeter waveform of the individual;
compute metric information associated with the instance of the pulse oximeter waveform; and
calculate a rate of rise or fall of the instance of the pulse oximeter waveform as a function of time.

2. The integrated circuit of claim 1, wherein the integrated circuit comprises a pulse oximeter.

3. The integrated circuit of claim 1, wherein, during the monitoring, the integrated circuit is configured to couple or attach to the individual.

4. The integrated circuit of claim 1, wherein the rate of rise or fall is expressed as a second derivative of an amplitude A of the instance of the pulse oximeter waveform, d²A/dt².

5. The integrated circuit of claim 4, wherein the second derivative of the amplitude of the instance of the pulse oximeter waveform corresponds to a trend in overall cardiac muscle function of the individual.

6. The integrated circuit of claim 4, wherein the integrated circuit is configured to identify a maximum value of the second derivative and its location in the instance of the pulse oximeter waveform.

7. The integrated circuit of claim 6, wherein the integrated circuit is configured to compare the maximum value and the location with another instance of the maximum value and the location obtained from a previous instance of the pulse oximeter waveforms; and
wherein, based at least in part on comparison, the integrated circuit is configured to evaluate a cardiac pumping function of the individual.

8. The integrated circuit of claim 4, wherein the second derivative corresponds to blood flow volume of the individual.

9. The integrated circuit of claim 4, wherein the second derivative corresponds to efficacy of a cardiac pumping function of the individual.

10. The integrated circuit of claim 1, wherein the integrated circuit is configured to compare a given instance of the pulse oximeter waveform with an immediately preceding instance of the pulse oximeter waveform to calculate the rate of rise or fall; and
wherein the given instance of the pulse oximeter waveform comprises the instance of the pulse oximeter waveform.

11. The integrated circuit of claim 1, wherein the integrated circuit is configured to provide a recommended remedial action based at least in part on the calculated rate of rise or fall of the instance of the pulse oximeter waveform.

12. An electronic device, comprising:
a light source; and
at least an integrated circuit, coupled to the light source, configured to:
monitor an instance of a pulse oximeter waveform of the individual;
compute metric information associated with the instance of the pulse oximeter waveform; and
calculate a rate of rise or fall of the instance of the pulse oximeter waveform as a function of time.

13. The electronic device of claim 12, wherein the electronic device comprises a pulse oximeter.

14. The electronic device of claim 12, wherein the rate of rise or fall is expressed as a second derivative of an amplitude A of the instance of the pulse oximeter waveform, d²A/dt².

15. The electronic device of claim 12, wherein the electronic device is configured to identify a maximum value of the second derivative and its location in the instance of the pulse oximeter waveform.

16. The integrated circuit of claim 15, wherein the electronic device is configured to compare the maximum value and the location with another instance of the maximum value and the location obtained from a previous instance of the pulse oximeter waveforms; and
wherein, based at least in part on comparison, the electronic device is configured to evaluate a cardiac pumping function of the individual.

17. A method for evaluating cardiac pumping function, comprising:
by an electronic device:
monitoring an instance of a pulse oximeter waveform of an individual;
computing metric information associated with the instance of the pulse oximeter waveform; and
calculating a rate of rise or fall of the instance of the pulse oximeter waveform as a function of time.

18. The method of claim 17, wherein the rate of rise or fall is expressed as a second derivative of an amplitude A of the instance of the pulse oximeter waveform, d²A/dt².

19. The method of claim 18, wherein the method comprises identifying a maximum value of the second derivative and its location in the instance of the pulse oximeter waveform.

20. The method of claim 19, wherein the method comprises:
comparing the maximum value and the location with another instance of the maximum value and the location obtained from a previous instance of the pulse oximeter waveforms; and
based at least in part on comparison, evaluating the cardiac pumping function of the individual.
